(19) United States
(12) Patent Application Publication
(10) Pub. No.: US 2024/0150690 A1
(43) Pub. Date: May 9, 2024

(54) Methods of Tissue Construct Fabrication Using a Modular Production System

(71) Applicant: Ralph L. Stirling, College Place, WA (US)

(72) Inventor: Ralph L. Stirling, College Place, WA (US)

(21) Appl. No.: 17/981,714

(22) Filed: Nov. 7, 2022

(51) Int. Cl. C12M 3/00; B33Y 30/00; C12M 1/12; C12M 1/26; C12N 11/00

(52) U.S. Cl. CPC C12M 21/08; B33Y 30/00; C12M 25/14; C12M 33/04; C12N 11/00

(57) ABSTRACT

A method for producing tissue constructs formed of one or more materials, dispensed or applied in patterned layers on two or more platforms, build plates or print surfaces by two or more stations, performing dispensing, mechanical modification, chemical modification, biological modification, or inspection operations. The platforms are positioned relative to the stations and station operations are directed by signals from a controller causing station operations on platforms to be performed simultaneously, for each patterned layer until all layers have been applied in sequence.

This application claims benefit of PPA 62/862,098, filed 2019 Jun. 16 by Ralph Stirling, which is incorporated by reference. This is a division of U.S. application Ser. No. 16/902,234, titled “Modular Tissue Construct and Additive Manufacturing Production System”, filed 2020 Jun. 15, now abandoned.

### BACKGROUND

A class of additive manufacturing systems for biomedical applications, called bioprinters or bioplotters, can produce tissue constructs for research and clinical repair or replacement of diseased or damaged tissue or organs. The tissue construct is built up by layers, incorporating materials that mimic the biological structures it is intended to replace or augment. Because natural biological structures are composed of an intricate network of materials of different sizes, mechanical properties, surface characteristics, and chemical attributes, multiple methods are often necessary for dispensing and patterning synthetic constructs.

### PRIOR ART

Conventional bioprinters have difficulty integrating widely different processes in a single machine. In addition, conventional bioprinters have been designed for small-scale laboratory and research use, and are incapable of scaling up to high-throughput production of synthetic tissue constructs. These conventional systems use only a few small syringes to hold material, which can only dispense material serially. They require frequent manual intervention to replace syringes, unclog dispensing tips, and remove finished constructs. Some prior art systems use mechanisms to switch syringes or dispensing tips, with one syringe in use at a time.

Conventional additive manufacturing systems (commonly known as “3D printers”) also suffer from serial processing limitations. Only one product or construct is produced at a time, and operations are performed sequentially while other dispensers are idle. A few additive manufacturing systems have two independent extruders on a single axis, but these either take turns dispensing material onto a single construct on the single build platform, or work in parallel to create just two identical objects side by side on the build platform.

### SUMMARY

The systems, methods, and devices of this disclosure each have several innovative aspects, no single one of which is solely responsible for the desirable attributes disclosed herein.

An implementation of the subject matter described in this disclosure is a modular system using multiple stations for dispensing materials in patterned layers on multiple platforms that move from station to station. Stations dispense materials simultaneously on multiple constructs. In addition to dispensing stations, inspection stations can be arranged to make careful documentation of intermediate layers during construct fabrication and to guide later operations. Tissue constructs are built up layer by layer as the platforms cycle through the stations. The layers may be planar, or may build up a 3-dimensional surface in some implementations.

System operation may be synchronous or asynchronous. A synchronous implementation positions each platform at each station for exactly the same amount of time, corresponding to the slowest operation. This is often achieved with an indexing turret, belt, or chain arrangement. An asynchronous implementation can bypass stations not needed for a particular layer, or have multiple identical stations for an operation that takes more time than the rest of the stations.

Advantages

Some implementations of the new concept have higher throughput because of the simultaneous operation of the multiple material dispenser stations. An implementation reduces the incidence of extrusion tip clogging because all stations are operating substantially simultaneously, without long idle periods.

At least one implementation provides in-process inspection between each patterned layer without reducing production throughput, since stations applying material continue to run simultaneously with inspection process stations.

At least one implementation reduces manual operations by automating the unloading of finished constructs and initiation of new constructs, leading to reduced risk of tissue construct contamination, greater efficiency, and lower labor costs.

Some implementations can produce much more complex tissue constructs than conventional bioprinters because stations can support very widely differing material deposition processes, such as polymer or hydrogel extrusion at one station and electrospinning at another.

At least one implementation can be easily reconfigured to add or remove material deposition operations by increasing or decreasing the number of stations in the process sequence.

### DESCRIPTION OF DRAWINGS

FIGS. 1A and 1B show implementations using an indexing turret.

FIG. 2 illustrates an implementation for producing tubular tissue constructs.

FIG. 3A-3D shows the station indexing sequence of a Cartesian actuator, stationary platform implementation.

FIG. 4 shows a system implementation similar to FIG. 3A-3D, but with stationary material dispensers and movable platforms.

FIG. 5 shows an implementation of a system dispensing multiple materials, inspecting each layer, and assembling a bioreactor chamber around the completed tissue construct.

FIG. 6 shows an implementation of a station using a serial kinematic manipulator or robot.

FIG. 7 illustrates a parallel link manipulator implementation of a dispensing station.

FIG. 8 shows a material dispensing manipulator station using a five-bar planar mechanism.

FIG. 9 shows a process for producing tissue constructs using the system of FIG. 5.

FIG. 10 shows an example of the patterns produced by each station of a three material implementation, with seven layers.

FIG. 11 illustrates the control logic of an implementation.

FIG. 12 illustrates an example of a prior art conventional bioprinter.

FIG. 13 shows an example material dispenser

### REFERENCE NUMBER LIST

100-199 platform elements
100 Turret type platform implementation
101 Platform in planar motor implementation
102 Platform in planar motor implementation
103 Platform in planar motor implementation
104 Platform in planar motor implementation
105 Platform in planar motor implementation
106 Platform in planar motor implementation
107 Platform in planar motor implementation
109 Platform and build plate in serial manipulator example
110 Rotation mechanism for tubular construct mandrels
111 Turret platform indexing mechanism
120 Tubular construct mandrel
121 Representative tubular construct
130 Build plate in conventional bioprinter
131 Platform and build plate 1 in cartesian implementation
132 Platform and build plate 2 in cartesian implementation
133 Platform and build plate 3 in cartesian implementation
140 Platform build plate in turret platform implementation
180 Bioreactor chamber build surface bottom
181 Bioreactor chamber wall
182 Bioreactor chamber top
200-299 station elements
200 Representative station for dispensing materials in a pattern
201 Station 1
202 Station 2
203 Station 3
204 Station 4
205 Station 5
206 Station 6
207 Station 7
211 X-axis actuator for a station
212 Y-axis actuator for a station
213 Z-axis actuator for a station
220 Five-bar planar manipulator, 3-degree-of-freedom type of station
221 Z-theta two-degree-of-freedom actuator station
231 First Z-theta two-degree-of-freedom actuator
232 Second Z-theta two-DOF actuator
233 First five-bar actuator link
234 Second five-bar actuator link
235 Third five-bar actuator link
236 Fourth five-bar actuator link
237 First revolute (non-powered) joint
238 Second revolute (non-powered) joint
239 Third revolute (non-powered) joint
241 X-axis actuator for conventional bioprinter
242 Y-axis actuator for conventional bioprinter
243 Z-axis actuator for conventional bioprinter
251 Serial manipulator rotary actuator joint 1
252 Serial manipulator rotary actuator joint 2
253 Serial manipulator rotary actuator joint 3
254 Serial manipulator rotary actuator joint 4
255 Serial manipulator rotary actuator joint 5
256 Serial manipulator rotary actuator joint 6
261 Rotary actuator for parallel link manipulator (one of three)
262 Ball joint (non-powered) for parallel link manipulator (one of twelve)
262 Revolute joint (non-powered) for parallel link manipulator (one of six)
280 Tray and placement mechanism for bioreactor chamber bottoms
281 Tray and placement mechanism for bioreactor chamber walls
282 Tray and placement mechanism for bioreactor chamber tops
300-399 controller elements
300 Block diagram of a control logic implementation for a single cycle
301 Outer loop of control logic, executed for each construct layer
302 Middle loop of control logic, executed for each system platform
303 Inner loop of control logic, executed for each system station
304 Retrieval or computation of motion sequence instructions
305 Initiation or execution of motion sequence
400-499 dispenser elements
401 Material extruder pump assembly
402 Syringe type extruder pump assembly
410 Material extrusion pump elements
411 Material extrusion nozzle
412 Material reservoir
413 Material reservoir support bracket
431 Extruder pump assembly for station 1 in cartesian implementation
432 Extruder pump assembly for station 2 in cartesian implementation
433 Extruder pump assembly for station 3 in cartesian implementation
500-599 construct diagram elements
500 Finished tissue construct on platform 1
501 First material, patterned as layer 1 by station 1
502 First material, patterned as layer 2 by station 1
503 First material, patterned as part of layer 3 by station 1
504 Second material, patterned as part of layer 3 by station 2
505 Third material, patterned as part of layer 3 by station 3
506 First material, patterned as part of layer 4 by station 1
507 Second material, patterned as part of layer 4 by station 2
508 Third material, patterned as part of layer 4 by station 3
509 First material, patterned as part of layer 5 by station 1
510 Second material, patterned as part of layer 5 by station 2
511 Third material, patterned as part of layer 5 by station 3
512 First material, patterned as layer 6 by station 1
513 First material, patterned as layer 7 by station 1
600-699 state diagram elements
600 Process
601 Extrude construct support material
602 Dispense first biomaterial at station 1
603 Dispense second biomaterial at station 2
604 Inspect current layer
605 Assemble bioreactor chamber around finished tissue construct
606 Remove bioreactor chamber
700-799 fixed system elements
700 System base
701 System base incorporating planar motor stator elements
703 Attachment point for parallel link manipulator
800-899 inspection station elements
801 Digital camera or digital microscope
802 Scanning probe microscope

### DETAILED DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

### PRIOR ART

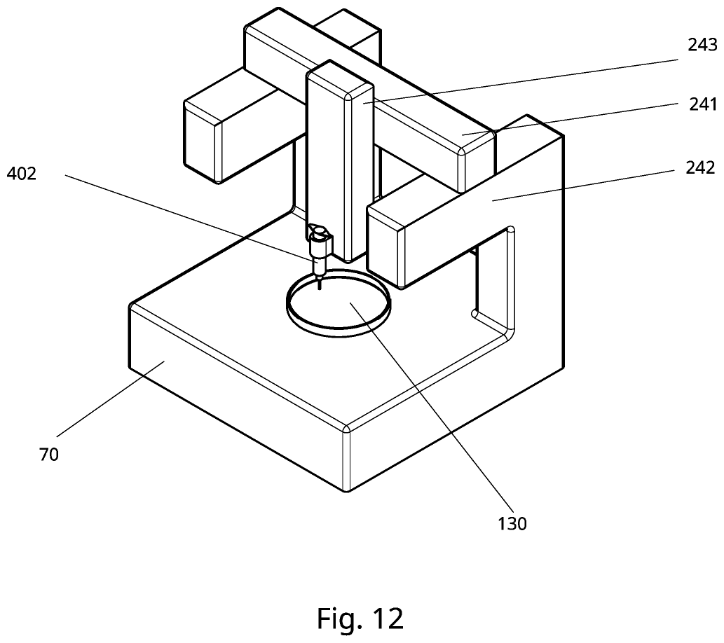

A typical conventional bioprinter is illustrated in FIG. 12. Most prior art bioprinters use a Cartesian 3-axis manipulator that moves a material dispenser 402, such as a syringe pump to pattern a single material type in layers on the stationary platform build plate 130. Some prior art bioprinters may be capable of switching between different material dispensers 402, and some may move the platform 130 in one or two axes while moving the dispenser 402 in the remaining axis or axes. When all the layers of a construct have been patterned on platform 130, a technician must manually remove the construct or the platform 130 from base 700 and prepare the system for a new construct.

Platforms

In some implementations of the subject matter in this disclosure, platforms move constructs under fabrication from station to station. The constructs may be identical or different. Each platform may have one or several constructs. Several arrangements for platforms are possible. Some implementations only move the construct between stations, and the station must provide all the motion for depositing material in a pattern. In other versions, the platform provides one or more axes of precision motion during material deposition, reducing the complexity of the station. In other implementations, platforms stay in specific locations, and station operations are brought to each platform in the appropriate sequence. The platforms in this version may have zero or more axes of motion while the station positioning mechanism supplies the remaining degrees of freedom to deposit material in the predetermined patterns.

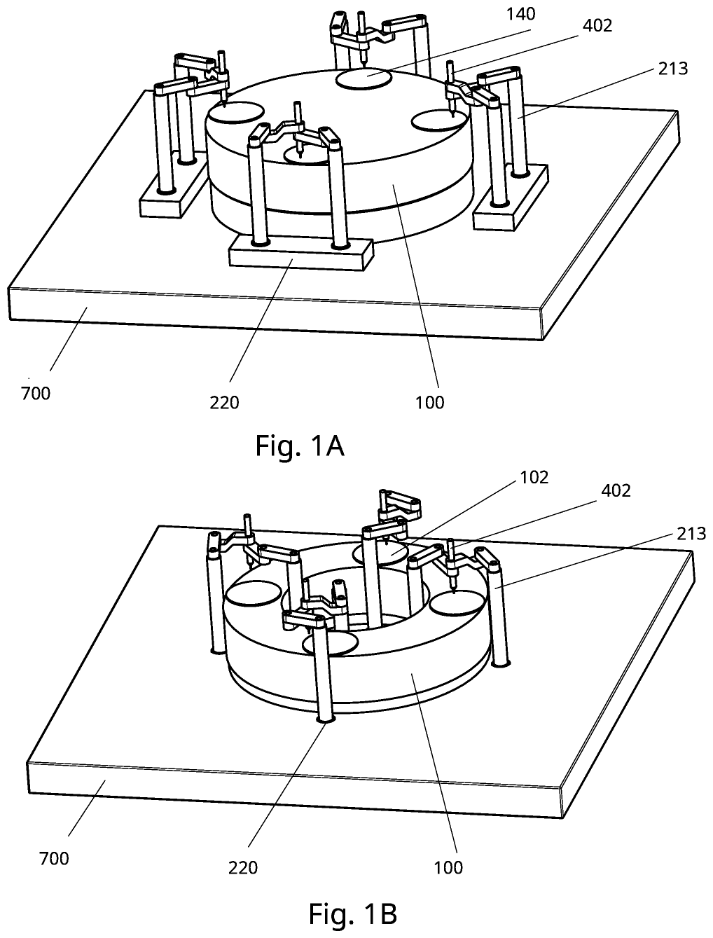

FIGS. 1A and 1B illustrate implementations that can dispense up to four different materials on each layer of a construct, and have four constructs in production simultaneously. Four stations 220 each provide three axes of motion for material dispensers 402. When each station 220 has completed its programmed pattern for a particular layer, the dispensers 402 are lifted clear of the constructs by station Z-axis actuators 213, and turret 100 indexes each platform build plate 140 to the next station. Each station 220 then applies a new pattern for the new layer. When all layers of a construct have been completed, an external mechanism, not shown, may remove the finished construct, and a new construct may be started on the now empty platform build plate 140 after the next index of turret 100. In the indexing turret implementations, station manipulators may be fixed outside the perimeter of the turret 100 as shown in FIG. 1A, straddle a ring-type turret 100 in FIG. 1B, or reside entirely inside a ring type turret 100 or above a disk type turret 100. Other types of station manipulators 220 may be used, such as serial link manipulators, Cartesian x-y-z manipulators, parallel link manipulators, or other kinematic mechanisms. In some implementations, the turret may rotate on a center bearing or pin or a slewing ring type of bearing. An implementation may have a removable turret for convenient cleaning or sterilization.

Rather than a turret, individual platforms may be connected with a precision pin and bearing, such that the collection of platforms forms a chain in another implementation. In versions of this disclosed subject matter, the chain may ride on or in a track affixed to the base, and may have driven or idler sprockets at two or more points. The chain can be passive, using a station's motion ability to advance to a new position, or actively move with a driven sprocket or other means of engaging an actuator to the chain. One version could use wire coils embedded in the base to provide electromagnetic force against magnets attached to the platform chain, forming a linear motor.

Platforms may also be positioned by articulated manipulators, either locked in place after cycling, or fully controlled at all times by the manipulators. These manipulators could be dedicated to each platform, or shared by multiple platforms to cycle between operations at stations. Another version could use station manipulators to provide platform cycling between station operations.

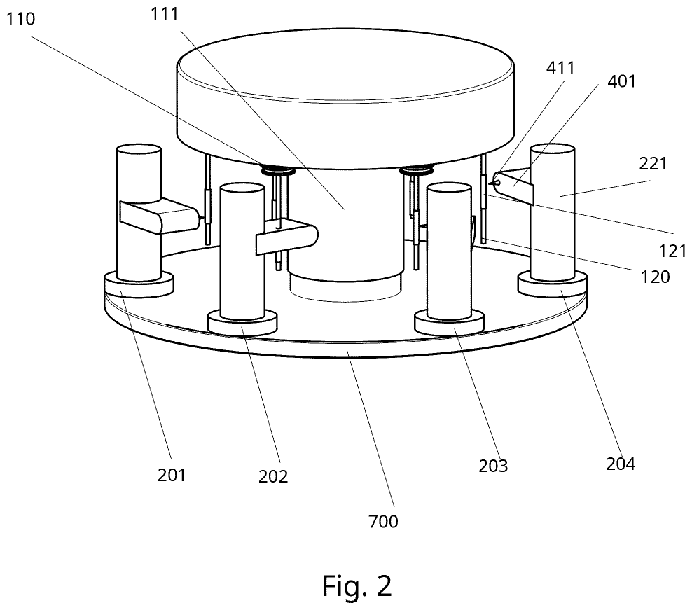

Platforms do not need to be planar. For example, an implementation for producing tubular constructs, such as small diameter vascular grafts or nerve conduits, could use hollow tubes or solid rods as build platforms, disposed at each station to pattern materials in layers around the circumference and length of the cylindrical build platform. FIG. 2 shows an implementation of a system for producing tubular constructs 121, using four material deposition stations 201-204. Each station 201-204 has two axes of motion; a vertical linear axis, and a rotary axis, provided by the Z-theta actuator 221. Material dispensers 401 are positioned relative to the cylindrical mandrel build platforms 120, which are rotated by mechanism 110 to pattern material dispensed from nozzles 411 in layers on mandrels 120.

Platforms may be completely passive, or may include features like electric charge, heating or cooling, other types of environmental control, vibration, ejection or locking mechanisms.

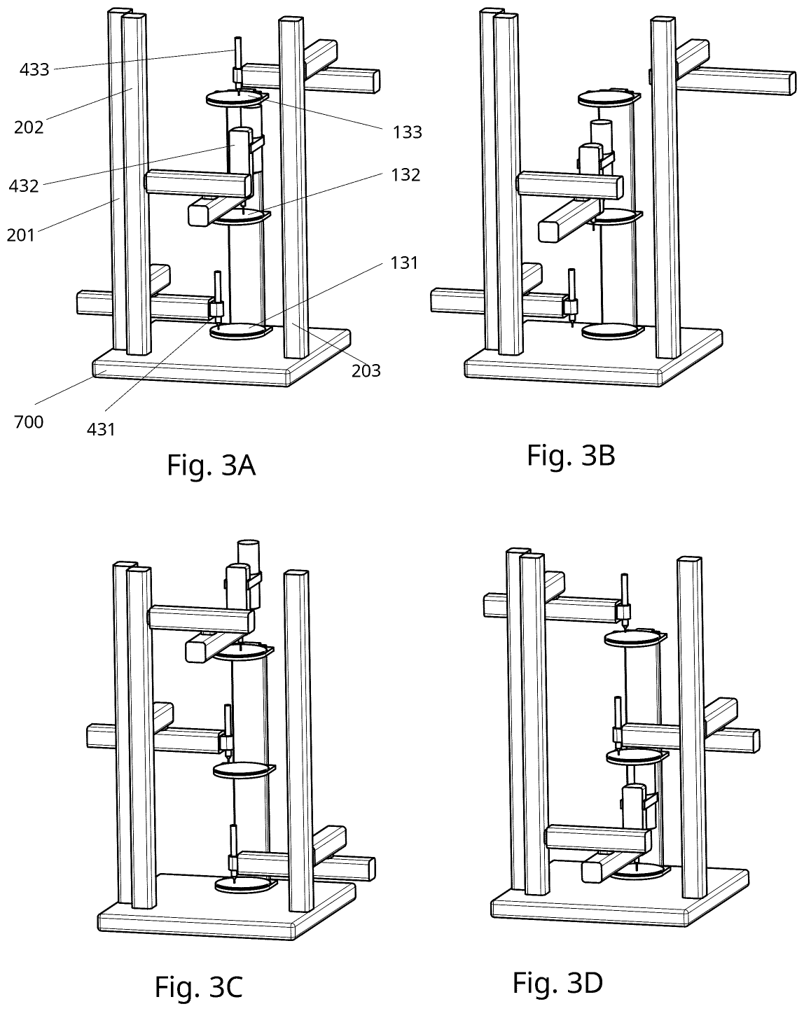

FIGS. 3A to 3D show an implementation with platform build plates 131, 132, and 133 fixed in a vertical arrangement. Each platform build plate will have a tissue construct formed layer by layer. The constructs may be identical, or may be unique. If the constructs are to be identical, then each station manipulator 201-203 executes each layer pattern three times, once for each platform 131-133. If the constructs are to be unique, then each station manipulator executes a different pattern for each layer on each platform 131-133. Cartesian manipulator stations 201, 202, and 203 move material dispensers 431, 432, and 433 relative to the fixed platforms 131-133 to dispense material for each construct layer. FIG. 3A shows dispenser 431 positioned to pattern material on the construct being built on platform 131, dispenser 432 positioned to pattern material on platform 132, and dispenser 433 positioned to pattern material on platform 133. FIG. 3B shows all three manipulators retracted after finishing the material patterning for the current layer on one platform, in preparation for moving to the arrangement illustrated in FIG. 3C. In FIG. 3C, dispenser 431 has moved up to platform 132, dispenser 432 has moved up to platform 133, and dispenser 433 has moved down to platform 131. Dispenser 431 will now apply the same pattern (if identical constructs), or a different pattern (for unique constructs). Following this layer, each station manipulator will retract again and reposition as shown in FIG. 3D.

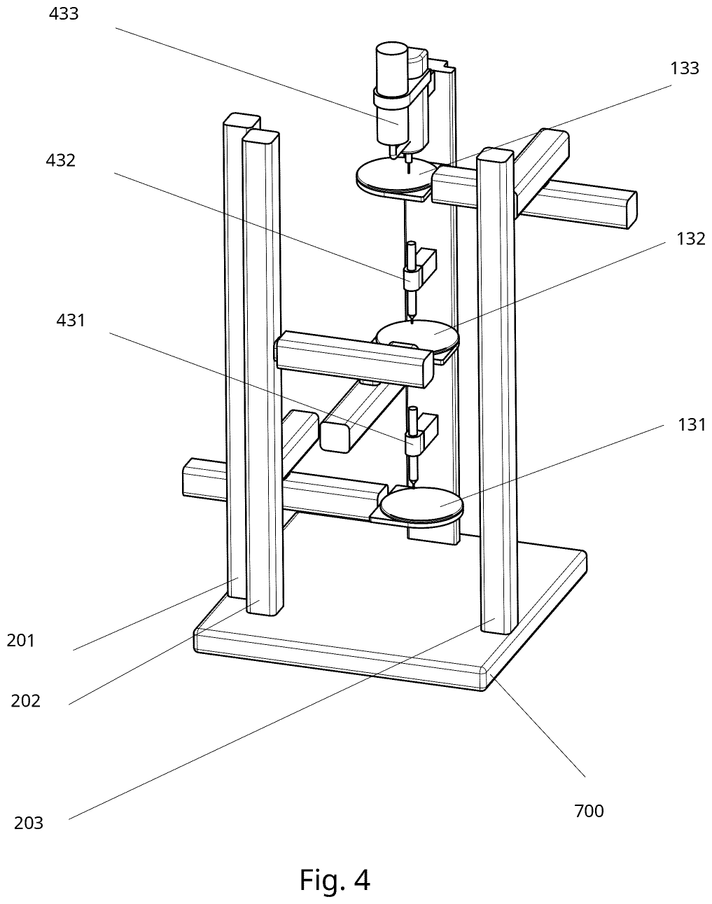

FIG. 4 shows another implementation using a similar arrangement as FIG. 3A-3D. The three Cartesian manipulators position the platform build plates 131-133 relative to fixed material dispensers 431-433. The movements for patterning each layer will be similar to those described for FIGS. 3A-3D. External mechanisms, not shown, could remove finished tissue constructs.

Independent Platforms

Platforms driven as independent linear motors, in one or more axes, can reduce the required complexity of stations by removing one or more degrees of freedom from the station motion in an implementation. Some implementations of independent linear motor platforms may have moving magnets attached to the platform, with stationary coils embedded or fixed relative to the base. Alternatively, coils may be incorporated in the platform, with stationary magnets affixed to the base. In this case, the motor coils in the platform may be energized by circuitry inside the platform that is powered from sliding electrical contacts or from inductive coupling from an alternating power source in the base. The platforms may be supported and constrained by a rail or track system, which may form a complete loop, or they may use magnetic levitation to hold a position above the base. Another alternative bearing arrangement is air bearings, in which the platform rides on an air cushion a small distance above the base or track.

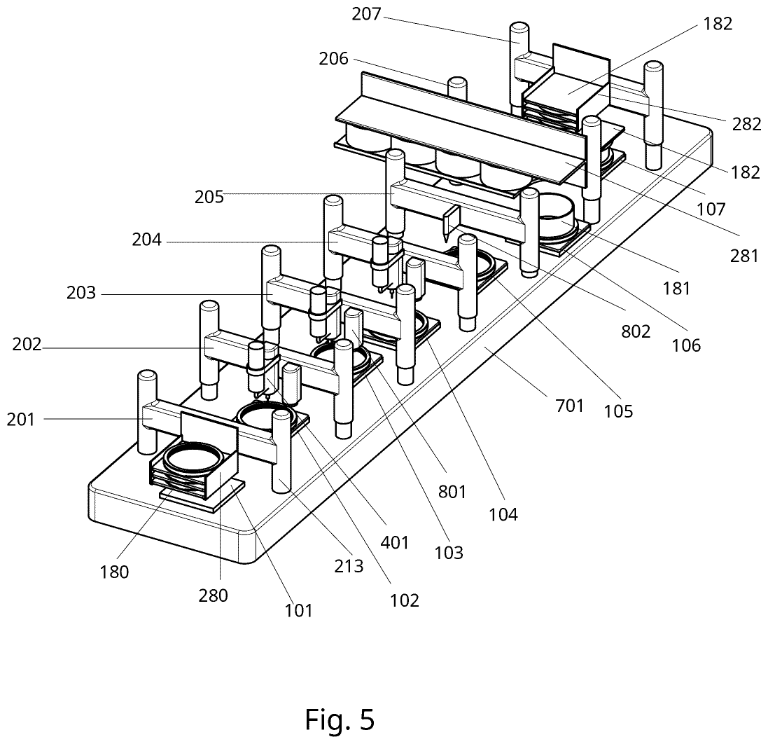

FIG. 5 illustrates an implementation based on a planar motor system. Stator coils are positioned in base 701 and controlled by the system controller (not shown) to magnetically position platforms 101 to 107, moving them between stations 201-207 and controlling the patterning of material dispensed by material dispensers 401 at stations 202-204. In the FIG. 5 system, the tissue constructs are deposited layer by layer on a bioreactor chamber bottom element 180. In the snapshot of the manufacturing process captured in FIG. 5, platform 101 is positioned to receive a new bioreactor chamber bottom 180 from storage mechanism 280 at station 201. Platforms 102, 103, and 104, carrying chamber bottoms 180 cycle through stations 202, 203, and 204, patterning materials from dispensers 401 on each layer. The construct on platform 105 is undergoing inspection at station 205 using a scanning probe or optical instrument 802. This inspection could take place every layer or less frequently. A finished construct on a chamber bottom 180 on platform 106 has a chamber wall element 181 pressed onto chamber bottom 180 fed from chamber wall dispenser element 281 at station 206. At station 207, a chamber top 182 is pressed onto chamber wall 181 by chamber top dispenser element 282. Finished tissue constructs sealed in chambers are removed from platforms by external mechanisms, not shown.

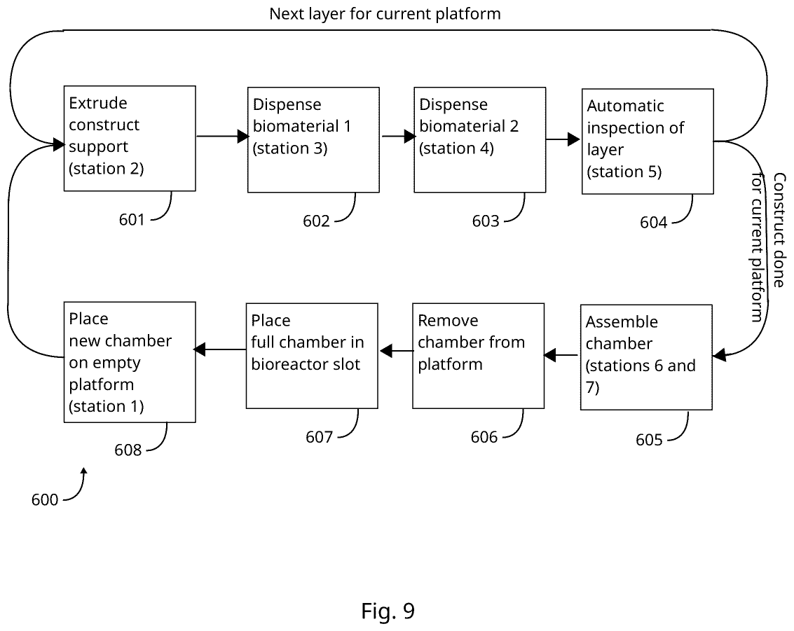

State diagram 600 in FIG. 9 illustrates an example process for the implementation of FIG. 5, viewed from the perspective of an individual platform. An empty chamber bottom is placed on the empty platform in block 608, then cycles through blocks 601, 602, 603, and 604, receiving material deposition patterns and inspection until all layers are complete. When all layers of the construct have been patterned, the chamber assembly is completed in block 605, and the external mechanism removes the completed chamber with construct in blocks 606 and 607.

Platform Print Surface

The tissue construct or scaffold may be printed directly on the platform surface in some implementations, which could be either smooth or textured, metal or plastic, or dispensed into a tray, well plate, or other chamber, either reusable or single-use in other implementations. The chamber could be a component that assembles or transfers readily to a bioreactor. The surface, tray, or chamber may have a conductive surface that can be selectively and automatically attached to a high-voltage circuit for electrodynamic deposition processes, either as ground, positive, or negative high voltage. The tissue construct, tray, well plate, or chamber may be automatically removed from a platform on completion of processing in the system. This could take place at a station or through some special mechanism.

Stations

Stations position material extruders or dispensers, inspection instruments, or mechanical manipulators relative to the platform, in some implementations. If the platform is moved into position and then locked in place, such as with a turret or chain arrangement, then stations that need to have a full range of motion must have at least three axes of motion. If the platform subsystem can provide one, two, or three degrees of motion in coordination with the station, then the station may have as few as zero degrees of freedom or axes of motion in some implementations.

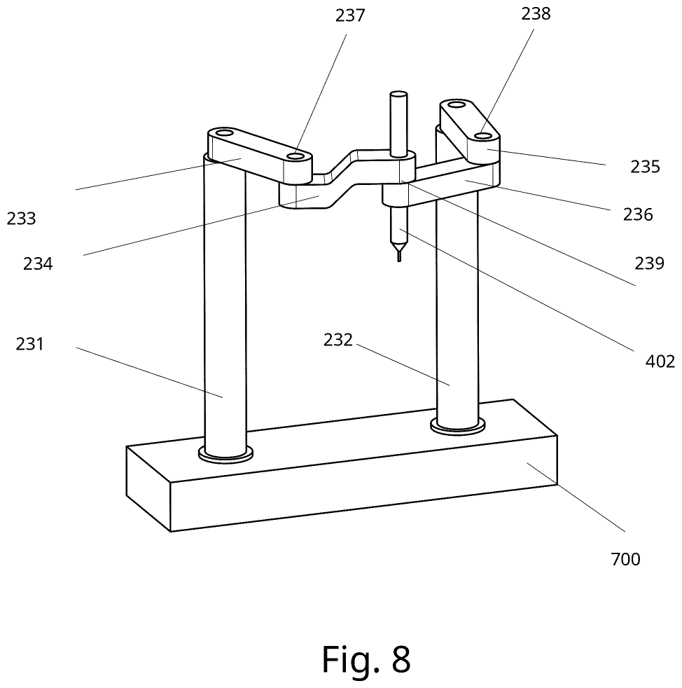

For example a turret platform system, such as those shown in FIGS. 1A and 1B, previously discussed, can be implemented with a planar manipulator with Z-axis actuators as shown in FIG. 8. Base 700 is the system base, as shown in both FIG. 8 and FIGS. 1A and 1B. Elements 231 and 232 are Z-theta actuators, which move vertically and rotate. Links 233, 234, 235, and 236, and non-powered revolute joints 237, 238, and 239 constitute a five-bar mechanism with two planar degrees of freedom. The material dispenser pump is concentric with joint 239. Because in at least some implementations, the material dispensing process is rotationally symmetric, the rotation of joint 239 at different plane positions will not affect the patterning process.

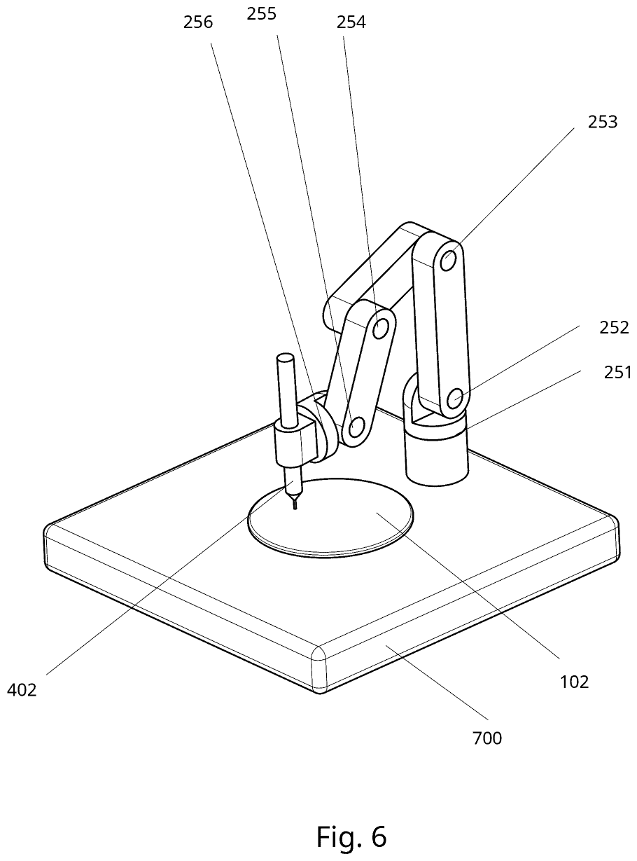

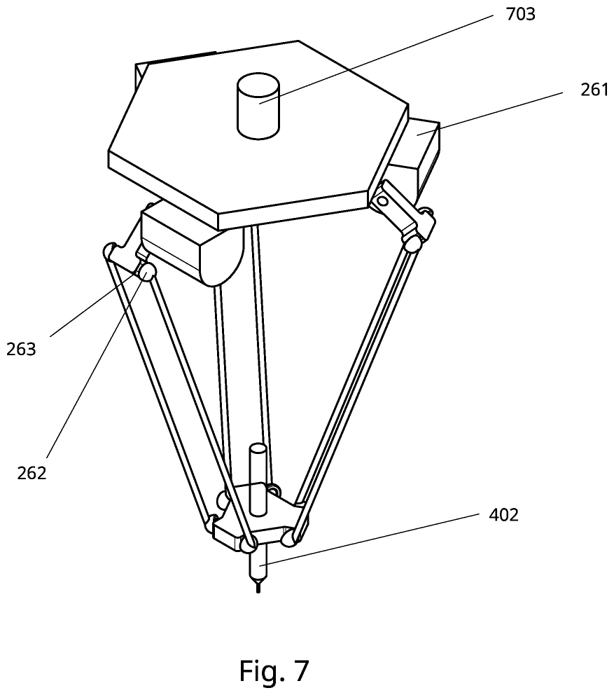

Another implementation of the system could use six-axis serial link robot manipulators to position material dispensers, such as the example shown in FIG. 6. The robot in FIG. 6 has a base 700 (which would be the turret in a turret platform system like FIG. 1A or 1B), build plate 109, rotary actuator joints 251-256, and a syringe pump dispenser 402. Alternatively, parallel link or delta robot manipulators, such as shown in FIG. 7, could position a syringe pump 402 for patterning material, using three rotary actuators 261, coupled through mechanical links and ball joints 262 (twelve total) and revolute joints 263 (six total).

An implementation of a planar x-y magnetic levitation platform system as shown in FIG. 5 (described above) would only need Z-axis vertical positioners for material dispensing stations.

Other implementations may use a variety of different manipulators within a single system. For example, a station that is dispensing material by electrospinning may not need any motion, as the patterning action takes place chaotically due to the asymmetries of the electric fields and the charged liquid polymer stream. Other stations in the same system might need one, two, three, or more degrees of freedom, depending on the nature of the dispensing process at each station. Additional degrees of freedom at a station beyond those kinematically necessary can reduce complexity of other components of the system or enable secondary operations, such as unloading constructs, in some implementations.

Materials Processed

The subject of this disclosure may dispense many different types of materials. Synthetic or natural polymers, such as polycaprolactone (PCL), polyurethane (PU), or collagen may be dissolved in appropriate solvents and dispensed in a viscous liquid form. Hydrogels of various compositions, with or without cells seeded may be dispensed. Additives such as melanin or hydroxyapatite may be incorporated in solutions. Polymers provided in a granular, powdered, or filament form may be liquified by melting and dispensed. Growth factors for different cell types may be selectively dispensed in the construct. Cells may be seeded directly into the tissue construct selectively. Soluble support material, such as sugar, may be extruded to create channels or internal cavities that are washed out in a post-processing step. Beads of growth factor or oxygen-supplying molecules may be deposited in the construct. Nanoparticles may be dispensed or incorporated in other materials. Because of the modular nature of the system, new materials not currently in use or discovered may be incorporated at a later time.

Material for a construct may be dispensed in many different ways. Stations interact with platforms to selectively dispense materials in precise quantities and rates onto the print surface and previous layers of material.

Solution Extrusion

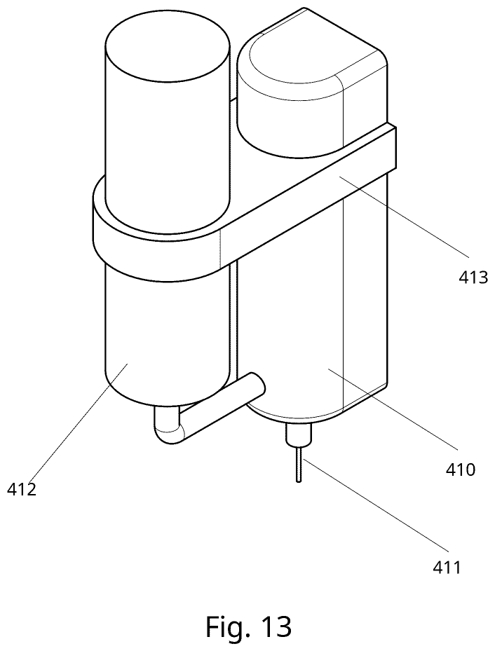

Dissolved polymers, which solidify upon drying, can be dispensed from syringes (compressed air actuated or mechanically motor actuated), peristaltic pumps, progressive cavity pumps, screw auger pumps, diaphragm pumps, or other pumping mechanisms currently known or developed in the future. Ink jet type print heads can dispense tiny droplets of liquid polymer using piezoelectric or electromagnetic actuators. Any of these dispensing techniques may be used in one or more different implementations of the subject matter of this disclosure. FIG. 13 illustrates an example implementation of an extruder capable of dispensing a large quantity of material over an extended period of time without attendance by a human operator.

Material reservoir 412, which may be pressurized in some implementations, allows liquid material to flow into the pump assembly 410, which extrudes the material through nozzle tip 411. Different implementations of pump assembly 410 might be progressive cavity, screw auger, peristaltic, diaphragm, or other types known to those with ordinary skill. Bracket 413, in some implementations, may keep the material cartridge 412 stable.

Melt Extrusion

Solid polymers, in the form of rods, filaments, granules, or powder, may be extruded through a heated nozzle by mechanical force or compressed air. The nozzle may be heated by resistance, induction, or laser heating.

UV Cure Extrusion

Liquid polymers with photoinitiator additives may be cured after extrusion by application of UV light by LED or laser.

Electrodynamic Deposition

Electrodynamic processes include electrospinning, electro writing, electrospray, and similar methods. Electrodynamic processes form micro or nanofibers from liquid polymer by application of a high electric field. The polymer may be liquified by either dissolving or melting. By appropriate selection of parameters such as viscosity, distance between dispensing tip and print surface, electric field strength, dispensing rate, and motion relative to the print surface, the nanofibers may be controlled in diameter, degree of alignment, and density. Magnetic fields may also be used to steer the nanofiber or selectively align fibers.

Direct Patterning

In addition to creating tissue constructs or scaffolds by relative motion of the station extrusion tip and the print surface, direct lithographic means can be utilized. UV optical lithography can be performed at a station by projecting patterns using programmable LCD masks or micromirror arrays with UV light onto a liquid polymer layer or pool with photoinitiator constituent. Uncured polymer may be removed by vacuum. Lasers of various wavelengths may also be used to modify the surface characteristics of previously formed tissue construct layers or to trim a tissue construct to a certain shape or size after formation.

Mechanical Manipulation

Pick and place stations may be used to remove finished tissue constructs or to place physical objects, such as electronic components, into constructs being fabricated. Heaters or chillers may be incorporated into stations to control viscosity or keep cells alive before seeding.

Bioreactor chamber print surfaces may incorporate mechanical elements to provide beneficial stress to seeded cells during the subsequent formation of a tissue construct.

Inspection Instruments

Stations may have inspection devices alongside other functions, or may be dedicated to particular inspection processes.

Digital Microscopy

Digital microscope cameras may be incorporated in many stations for purposes of alignment of platforms at each station, in some implementations. One or more optical targets on the platform can be imaged to establish a reference frame for subsequent material patterning at that station. The digital microscope can also image the tissue construct for quality control purposes at each layer and station.

Scanning In

Since there is already an accurate, high resolution scanning motion between station and platform, scanning processes such as scanning optical microscopy, atomic force microscopy, or other scanning probe microscopy methods may be incorporated in specialized stations in some implementations. These techniques can be used to verify material mechanical properties during the tissue construct formation.

Tissue Construct Formation

The relative motion of material extruders at stations and platform print surfaces deposit a patterned layer of material on the print surface or on the previous layer. The layering may be strictly two-dimensional, with no vertical motion during extrusion, or three-dimensional motions during extrusion may take place to dispense material over a built-up area on the construct.

An implementation of the subject of this disclosure may be fabricating identical tissue constructs on each platform, or may be forming different constructs on each platform. Non-identical constructs may be started on an empty platform at any time, and finished constructs may be removed independently of unfinished constructs. When a construct is finished, it may be moved to a removal station that picks the construct or entire print surface or chamber and moves it out of the production system, freeing up the platform for a new construct.

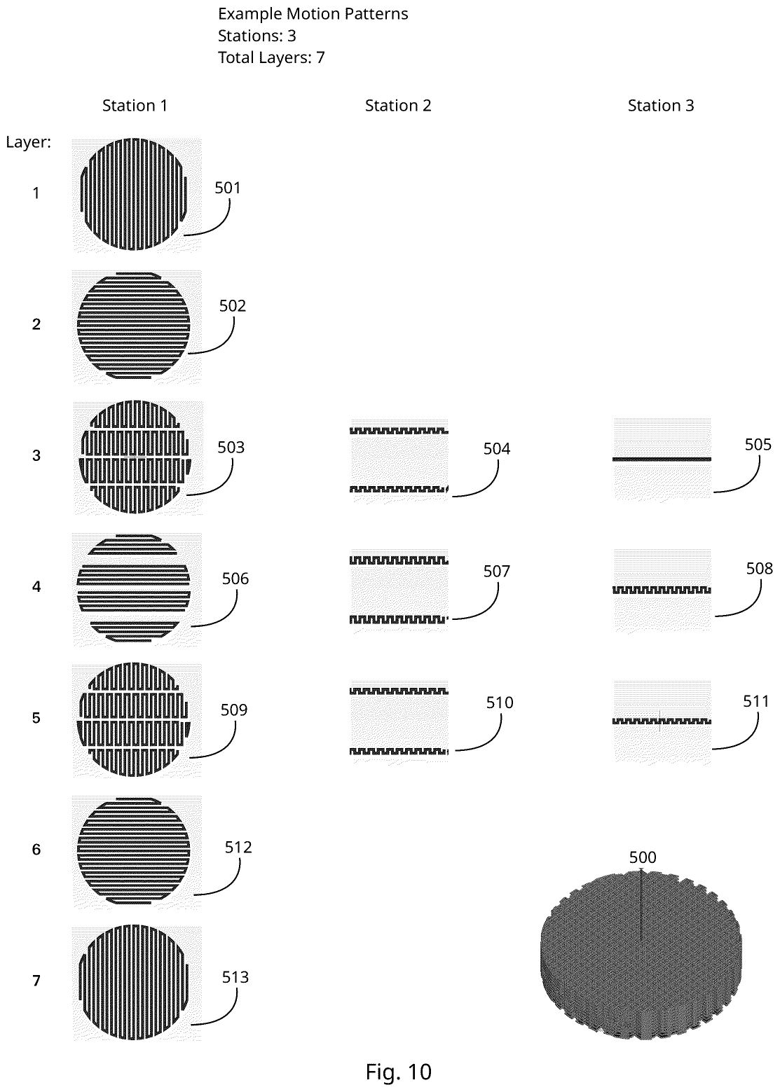

FIG. 10 gives a simple illustration of an example implementation using three dispensing stations to create a construct 500 of seven layer patterns 501-513. Layer 1 contains a single material, dispensed at station #1 in pattern 501. Layer 2 is the same material dispensed on top of layer 1, but in pattern 502. Layer 3 first receives station #1 material in pattern 503 on top of layer 2, then receives station #2 material in pattern 504 in the gaps in pattern 503. Finally, station #3 dispenses material in pattern 505 in the remaining gap on layer 3. Layer 4 dispenses pattern 506 at station #1, then pattern 507 at station #2, and pattern 508 at station #3. Layer 5 combines pattern 509 at station #1, pattern 510 at station #2, and pattern 511 at station #3. Layers 6 and 7 only use station #1, with patterns 512 and 513. This tissue construct could be produced on any of the system implementations discussed, as well as other configurations. At least three platform build plates would be used, producing three identical tissue constructs 500 at the end of a cycle.

Software

Simultaneous Station Action

Stations have independent, but coordinated control, such that multiple types of material or areas of a layer are extruded simultaneously on different platforms in some implementations. Platforms cycle through the stations until all layers have been completed on all platforms. The simultaneous station activity can increase throughput substantially, and reduce the incidence of clogged dispensing tips, a frequent problem with conventional multi-material, single station, single platform systems. Sterility can be much easier to maintain in some implementations, as far less human touch or manual intervention is required.

Each station has a motion program or sequence for each layer of each platform. If a station has a much shorter activity duration than average, the program may introduce small delays at intervals so the total activity duration is close to that of the longest station duration to reduce incidence of tip clogging.

Top Level Software

The top level control software for the system monitors completion of station motion programs, directs platform movement between stations, starts and stops the system, and monitors deposition quality and system anomalies. The motion planning software for each station and layer may be precomputed for all constructs and stored in readiness for each station and layer, or it may be generated in real time by a central control computer, or distributed control computers associated with each station. If the control is decentralized, then the top level software is responsible for communicating motion plans to each station controller, in some implementations.

Environmental Control

The system, in some implementations, may be built in a form factor to fit inside a standard biological safety cabinet, or, in other implementations, it may be fully enclosed with appropriate levels of particle filtration on inward airflow, and chemical filtration on exhaust air. If live cells are incorporated into the tissue construct, the system may be operated inside an incubator that controls temperature and CO2 gas concentration for optimum cell health.

Tissue constructs, in some implementations, must be kept uncontaminated from the point of manufacture to the point of use. If the construct incorporates live cells, then the proper environmental conditions must be maintained during the entire transport process to the point of use. This may be accomplished with some type of bioreactor system. An implementation of the subject matter of this disclosure, can place completed tissue constructs in an appropriate modular bioreactor chamber at the last station, such that no human handling of the living tissue construct is necessary before point of use. A version of this could attach or print a bar code or other identifier on each bioreactor chamber for purposes of tracking the tissue construct, which may be unique or tailored for a particular patient.

Controllers

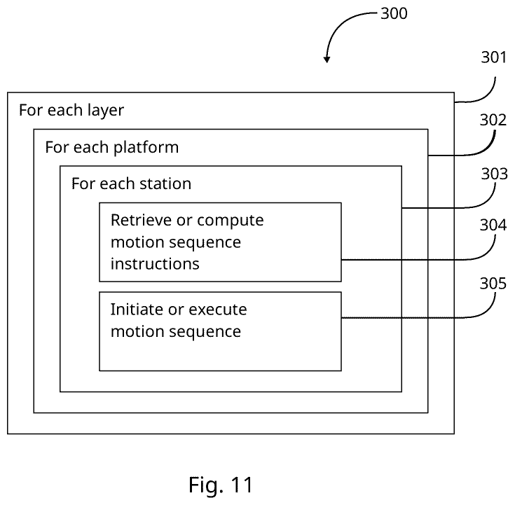

In some implementations, complex systems, such as implementations of the subject matter of this disclosure, can utilize programmable control to sequence some or all of the associated operations. FIG. 11 block diagram 300 shows one example of the high-level control steps of an implementation. For purposes of the example, let the tissue construct being produced contain N layers, and the system implementation involving M stations and P platforms. The logic 300 is executed for each cycle of the system, producing P constructs each cycle. The outer loop of logic, block 301, is executed once for each layer, or N times per cycle. During each layer 301, the logic then cycles through each platform, block 302. For each station in the system, represented by logic inner loop block 303, a sequence of motion instructions is retrieved or computed (block 304), and then initiated or executed (block 305). These motion instructions could control the positioning of platforms, stations, and dispensing of material or action of other elements. Block 303 is happening in parallel at all stations simultaneously in some implementations. In some implementations, block 302 is happening in parallel at all platforms.

The present disclosure describes various features, no single one of which is solely responsible for the benefits described herein. It will be understood that various features described herein may be combined, modified, or omitted, as would be apparent to one of ordinary skill. Other combinations and sub-combinations than those specifically described herein will be apparent to one of ordinary skill, and are intended to form a part of this disclosure. Various methods are described herein in connection with various flowchart steps and/or phases. It will be understood that in many cases, certain steps and/or phases may be combined together such that multiple steps and/or phases shown in the flowcharts can be performed as a single step and/or phase. Also, certain steps and/or phases can be broken into additional sub-components to be performed separately. In some instances, the order of the steps and/or phases can be rearranged and certain steps and/or phases may be omitted entirely. Also, the methods described herein are to be understood to be open-ended, such that additional steps and/or phases to those shown and described herein can also be performed.

Some aspects of the systems and methods described herein can advantageously be implemented using, for example, computer software, hardware, firmware, or any combination of computer software, hardware, and firmware. Computer software can comprise computer executable code stored in a computer readable medium (e.g., non-transitory computer readable medium) that, when executed, performs the functions described herein. In some embodiments, computer-executable code is executed by one or more general purpose computer processors. A skilled artisan will appreciate, in light of this disclosure, that any feature or function that can be implemented using software to be executed on a general purpose computer can also be implemented using a different combination of hardware, software, or firmware. For example, such a module can be implemented completely in hardware using a combination of integrated circuits. Alternatively or additionally, such a feature or function can be implemented completely or partially using specialized computers designed to perform the particular functions described herein rather than by general purpose computers.

Multiple distributed computing devices can be substituted for any one computing device described herein. In such distributed embodiments, the functions of the one computing device are distributed (e.g., over a network) such that some functions are performed on each of the distributed computing devices.

Some embodiments may be described with reference to equations, algorithms, and/or flowchart illustrations. These methods may be implemented using computer program instructions executable on one or more computers. These methods may also be implemented as computer program products either separately, or as a component of an apparatus or system. In this regard, each equation, algorithm, block, or step of a flowchart, and combinations thereof, may be implemented by hardware, firmware, and/or software including one or more computer program instructions embodied in computer-readable program code logic. As will be appreciated, any such computer program instructions may be loaded onto one or more computers, including without limitation a general purpose computer or special purpose computer, or other programmable processing apparatus to produce a machine, such that the computer program instructions which execute on the computer(s) or other programmable processing device(s) implement the functions specified in the equations, algorithms, and/or flowcharts. It will also be understood that each equation, algorithm, and/or block in flowchart illustrations, and combinations thereof, may be implemented by special purpose hardware-based computer systems which perform the specified functions or steps, or combinations of special purpose hardware and computer-readable program code logic means.

Furthermore, computer program instructions, such as embodied in computer-readable program code logic, may also be stored in a computer readable memory (e.g., a non-transitory computer readable medium) that can direct one or more computers or other programmable processing devices to function in a particular manner, such that the instructions stored in the computer-readable memory implement the function(s) specified in the block(s) of the flowchart(s). The computer program instructions may also be loaded onto one or more computers or other programmable computing devices to cause a series of operational steps to be performed on the one or more computers or other programmable computing devices to produce a computer-implemented process such that the instructions which execute on the computer or other programmable processing apparatus provide steps for implementing the functions specified in the equation(s), algorithm(s), and/or block(s) of the flowchart(s).

Some or all of the methods and tasks described herein may be performed and fully automated by a computer system. The computer system may, in some cases, include multiple distinct computers or computing devices (e.g., physical servers, workstations, storage arrays, etc.) that communicate and interoperate over a network to perform the described functions. Each such computing device typically includes a processor (or multiple processors) that executes program instructions or modules stored in a memory or other non-transitory computer-readable storage medium or device. The various functions disclosed herein may be embodied in such program instructions, although some or all of the disclosed functions may alternatively be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list. The word “exemplary” is used exclusively herein to mean “serving as an example, instance, or illustration.” Any implementation described herein as “exemplary” is not necessarily to be construed as preferred or advantageous over other implementations.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings of the invention provided herein can be applied to other methods and systems, and are not limited to the methods and systems described above, and elements and acts of the various embodiments described above can be combined to provide further embodiments. Accordingly, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A method for producing layered tissue constructs, comprising a controller, a set of platforms, and a set of stations;
the controller generating a first control signal to result in platforms indexing between stations;
generating additional control signals to position each platform relative to each station;
generating additional control signals to result in material dispensed at one or more stations in defined patterns simultaneously;
until all platforms have sequenced through all stations for all layers as required,
forming complete tissue constructs on each platform.

19. Method of claim 18, where the controller acquires inspection information from one or more stations related to the partially formed or finished tissue constructs.

20. A method of producing layered tissue constructs, carried out by a computer, comprising steps:
positioning platforms in relation to stations;
directing one or more stations to perform material dispensing operations for a layer of a tissue construct on each platform;
and sequencing the platforms and stations to successively form all layers of all tissue constructs, patterns and sequences for depositing layers computed or recalled from computer memory.

21. Method of claim 20, with the computer acquiring inspection information from one or more stations related to the partially formed or finished tissue constructs.

22. A method for producing tissue constructs formed of one or more materials,
dispensed or applied in patterned layers
on build plates or print surfaces on each of two or more platforms,
sequenced or indexed through two or more stations, the method comprising:
performing dispensing, mechanical modification, chemical modification, biological modification, or inspection operations;
operations of stations and relative positioning of platforms and stations directed by signals from a controller; and
causing station operations on platforms to be performed simultaneously,
for each patterned layer until all layers have been applied in sequence to all tissue constructs on all platforms.

23. Method of claim 22, where the sequencing of platforms through stations is performed synchronously.

24. Method of claim 22, where the sequencing of platforms through stations is performed asynchronously.

25. Method of claim 22, where the stations are held in stable positions, while the platforms are positioned relative to stations.

26. Method of claim 22, where the platforms are held in stable positions, while the stations are positioned relative to platforms.
